(19) United States
(12) Patent Application Publication
(10) Pub. No.: US 2018/0316243 A1
(43) Pub. Date: Nov. 1, 2018

(54) ROTOR FOR A PUMP, PUMP, AND ASSEMBLY

(71) Applicant: Berlin Heart GmbH, Berlin (DE)

(72) Inventors: Heiko Gundlach, Berlin (DE); Kim Peter Winterwerber, Berlin (DE)

(73) Assignee: Berlin Heart GmbH, Berlin (DE)

(21) Appl. No.: 15/768,357

(22) PCT Filed: Oct. 12, 2016
(86) PCT No.: PCT/EP2016/074487

§ 371 Date: Apr. 13, 2018

(30) Foreign Application Priority Data
Oct. 13, 2015 (EP) 15189562.0

(51) Int. Cl. H02K 7/09; H02K 7/14; H02K 15/16; F04D 3/02; F04D 13/06; A61M 1/10

(52) U.S. Cl. CPC H02K 7/09; H02K 7/14; A61M 1/1015; F04D 3/02; F04D 13/06; H02K 15/16

(57) ABSTRACT

A motor for a pump is provided with a radially outer stator and a radially inner rotor. The radially outer stator having a laminated core. The rotor is mounted in two magnetic axial bearings distanced from one another in an axial direction of the rotor. A position of the stator relative to a reference surface, against which a component rests, is established exclusively by a single positioning element arranged between the laminated core of the stator and the reference surface. A stationary part of a first magnetic axial bearing of the two magnetic axial bearings is integrated in the component.

### CROSS-REFERENCE TO RELATED APPLICATIONS

This application is a 371 nationalization of international patent application PCT/EP2016/074487 filed Oct. 12, 2016, which claims priority under 35 USC § 119 to European patent application 15189562.0 filed Oct. 13, 2015. The entire contents of each of the above-identified applications are hereby incorporated by reference.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 shows an axial pump with tangential outlet in a perspective view from outside;

FIG. 2 shows a longitudinal section through an axial pump with tangential outlet similarly to that shown in FIG. 1, but without housing; and

FIG. 3 shows a perspective view of a part (cut lengthwise) of the pump from FIG. 2, also without housing.

### DETAILED DESCRIPTION

What is proposed is a new type motor for a pump, and a pump comprising a motor of this kind, with the innovation lying in the field of mechanical engineering and mechanics or electromechanics. One possible application of the new motor or the pump lies for example in the field of conveying sensitive liquids.

New types of rotor pumps that can be economically produced and can be easily controlled and are maintenance-free and allow liquids to be conveyed continuously in a gentle manner are currently being used in many fields of application. Here, a maintenance-free mounting of the rotor that experiences extremely low levels of friction should be achieved at the same time. To this end, magnetic bearings are being used increasingly, which, amongst other things, can effectively intercept axial shear forces acting on the rotor during operation. However, the use of magnetic bearings of this kind, in particular when the rotor is to be supported in two magnetic bearings of this kind, presupposes a high level of precision of the manufacture and assembly of the individual parts and additionally the possibility to control the axial bearing components within a range limited by manufacturing tolerances as applicable.

The design of a motor and/or a pump that can be constructed with minimal manufacturing and assembly tolerances is desired.

In order to achieve the object, what is proposed is a motor for a pump having a radially outer stator, which has a laminated core, and having a radially inner rotor, wherein the rotor is mounted axially with respect to a pump tube in two magnetic axial bearings distanced from one another. In order to achieve the object, this motor additionally has the features that the position of a laminated core of the stator relative to a reference surface, against which a component rests, in which component the stationary part of a first axial bearing is integrated, is established decisively, in particular exclusively, by a single positioning element (an element arranged between a laminated core of the stator and the reference surface). The distance between the laminated core of the stator and the stationary bearings determines the axial position of the rotor.

In the case of the described motor it is a matter of establishing the position of the stator as easily and precisely as possible relative to the stationary bearing part of at least one magnetic axial bearing. The axial movement range of the rotor, which can move in the stator some way in the axial direction during operation, is thus also largely established. The stator can be positioned in such a way that its axial centre is arranged at a defined distance between two axial bearings provided in the axial direction on either side of the rotor. Thus, a certain play remains for the rotor for operation on either side in the axial direction, for example as considered from a central resting position in the stator.

The provided control system, by suitable control of one or more control coils, influences the magnetic circuit for axial bearing for example such that the rotor is located in the position where the axial forces cancel one another out exactly in both directions.

The rotor, more precisely in particular the bearing magnets integrated in the rotor and fixedly connected thereto, is/are attracted axially on both sides by stationary bearing magnets, wherein the overall force acting on the rotor is dependent on the magnet distance in the axial bearings, in such a way that the rotor, upon leaving this position, would be drawn directly to one of the two bearings without countermeasures (control system with control coil).

The axial bearing total gap permits only a specific maximum shift in the axial direction, which is effective at a maximum permissible rotational speed at a given axially acting fluid counterpressure. In order to be able to operate the pump at a sufficiently high rotational speed, the tolerances of the overall arrangement in the axial direction should be minimised.

When the rotor is released from the axial bearing that attracted the rotor as far as it can go prior to commissioning (generally only at the time of calibration and at the time of pump start-up), the coil, which is also provided for control of the axial bearing, is energised much more strongly than in normal operation, up to a specified limit. Optimised tolerances also mean that the rotor is released with lower currents through the control coil.

During operation, the current through the control coil and the position of the rotor are measured continuously. The current is continually corrected by means of a complex control system and under consideration of the rotor position.

The necessary minimisation of the mechanical tolerances is provided in that the stator of the motor is directly aligned with a reference surface to the greatest possible extent, with a component also being aligned therewith at the same time, the stationary part of a first axial bearing being integrated in said component. The stator is aligned with the reference surface by means of a single positioning element arranged between a laminated core of the stator and the reference surface. Only a single part affected by tolerances is thus provided there. The laminated core, against which the positioning element directly rests, is decisive for the position of the stator, such that a potting compound of the stator is removed from the tolerance chain. Corresponding assembly gaps can thus be provided between the potting compound of the stator and parts resting thereagainst.

The positioning element can consist of a number of different elements, also made of different materials, which in particular can be fixedly connected to one another, but does not have any further mechanical tolerances.

For example, it can be provided that the positioning element guides the magnetic reflux from the stator laminated core to the stationary part of the first axial bearing. For example, the positioning element can be a tubular element which is coaxial with the rotor and which is made of a material that guides the magnetic flux with low resistance. A reflux element of this kind is required in many cases in order to suitably guide the magnetic flux through the axial bearing which flows back axially through the rotor, from there into the stator and radially outwardly.

It can also be provided that the positioning element guiding the magnetic reflux of the first magnetic axial bearing rests against the laminated core of the stator and against the reference surface without gaps.

The positioning element is provided as a single component, wherein this can be formed of one piece from homogeneous material, but also can consist of a plurality of parts joined fixedly together. Here, it is merely advantageous that it is joined together as a component so fixedly that it has only a single tolerance value, in particular with respect to the axial dimensions. The positioning element on the whole can be constructed homogeneously with respect to the material properties, however it can also have inhomogeneities, for example due to layers that are less magnetically conductive.

A motor of the above-described type specifically can be configured for example so that a first axial bearing has a first stationary magnetically active element (a magnetically active element can be one or more magnets, one or more reflux parts, or a combination thereof and also can be supplemented by non-magnetic parts), which interacts magnetically with a first magnetically active element of the rotor, wherein the magnetic circuit is closed by the first magnetically active element of the rotor via the first stationary magnetically active element, optionally a connection piece-like or pin-like magnetically active element with flange (for example a reflux part), a disc-like magnetically active element forming the reference surface (for example a reflux part), against which the flange of the connection piece or pin rests flat, and a hollow-cylindrical magnetically active positioning element (for example a reflux part) to the stator, and from there to the rotor, and wherein the laminated core of the stator rests against the hollow-cylindrical magnetically active positioning element without gaps in the axial direction, and the latter rests against the reference surface of the disc-like magnetically active element without gaps in the axial direction. A second axial bearing can be constructed differently.

The entire magnetic circuit of at least one of the axial bearings is therefore closed by the magnetically active parts of the bearing on the stator and rotor side, the rotor itself, the laminated core of the stator, and the reflux elements, consisting of the positioning element, the disc-like element, and the connection piece or pin with flange.

It can additionally also be provided that the rigidity of at least one axial bearing for balancing of the two axial bearings within a generally narrow range is possible by exchange or omission of one or more parts. The connection piece or pin with flange can thus have a gap from the adjacent magnetic active element. The width of the gap (possibly determined by the length of the connection piece or pin) influences the rigidity of the axial bearing in question.

The balancing of the two axial bearings is also possible by suitable positioning of the connection piece or pin in the axial direction, in that it is displaceable in as finely graduated a way as possible, for example in a thread in the axial direction. An adjustment can also be performed by the selection of a suitable adjustment piece from a predefined selection of different adjustment pieces with different reflux properties. However, adjustment can also be provided by means of a slot, which can be closed or bridged by rotation of a counter piece.

In the structure of the motor it can also be provided that one or more assembly gaps is/are provided between the stator and the elements of the pump housing, in particular the delimitation elements of a flow chamber axially adjoining the stator. In particular, the potted elements of the stator should be arranged at a distance in the axial direction from the delimitation elements/walls of the flow chamber, with the function of providing an assembly gap for tolerance compensation.

It can additionally be provided that the disc-like magnetically active element has a diameter which corresponds approximately to the outer diameter of the stator or which is between the inner diameter and the outer diameter of the stator. In particular when the positioning element is tubular or partially tubular and has a corresponding diameter, the magnetic reflux elements can thus guide the magnetic flux of the axial bearing around a flow chamber and close the magnetic circuit. For example, the hollow-cylindrical magnetically active positioning element thus surrounds a flow chamber with tangential outlet axially adjoining the stator.

In an advantageous embodiment, the stationary part of the bearing can be fixed relative to the pump housing, in particular fixedly connected thereto, by means of the component in which the stationary part of the first axial bearing is integrated.

Apart from the above-described motor for a pump, a new pump shall also be described here, which is characterised by a motor of the above-described type, wherein in particular the rotor, in the annular conveying chamber between the rotor and the stator, carries conveying elements for conveying a fluid.

The pump is usually formed as a rotor pump, wherein the rotor of the motor can directly carry conveying elements for conveying a fluid. The liquid can flow through an annular conveying chamber between a hub (rotor or guide vane) and pump tube (stator). A flow guide vane (held in the pump tube in a spoke-like manner) or a flow chamber with tangential outlet can be provided axially adjoining the rotor. The stationary and rotating part of the axial bearing are usually arranged such that the magnetically active parts do not come into contact with the conveyed liquid. The bearing magnets are for this purpose covered by covers which form part of the housing of the flow chamber or a flow guide vane.

Additionally to a motor and a pump of the above-described type, a method for assembling a motor and/or a pump shall also be presented, said method being characterised in that the reflux parts and the stator of the motor are placed in the pump housing and in that the rotor is then brought by control of the axial bearings into a stabilised position, and in that for calibration the location of the stabilised position is ascertained, in particular in the axial direction with respect to the axial bearing, when the motor is not turning.

The position can be detected by means of sensors, in particular eddy current sensors.

If, at the time of this trial-start-up of the pump, the location of the position of the rotor is satisfactory, the pump can be ultimately started without further ado. A desired shifting of the rotor position can also be provided by use of a suitable adjustment piece in the form of a magnetically active connection piece or pin of the above-described type.

It can be provided that, once the desired position has been ascertained with turning or non-turning motor during the testing, a magnetically active connection piece or pin is introduced into the disc-like magnetically active element or is swapped with a connection piece or pin located there previously. In this way, the greatest possible field of use is achieved in respect of the flow rate and fluctuations of the flow rate.

In the implementation of the above-described ideas, a group of pumps of the above-described type, which are characterised in that at least two of the pumps have different connection pieces or pins in respect of dimensions or magnetic reflux properties, is thus also provided.

Exemplary embodiments of the new, above-presented motor and a corresponding pump will be explained in greater detail hereinafter and presented on the basis of figures of a drawing.

In the drawing:

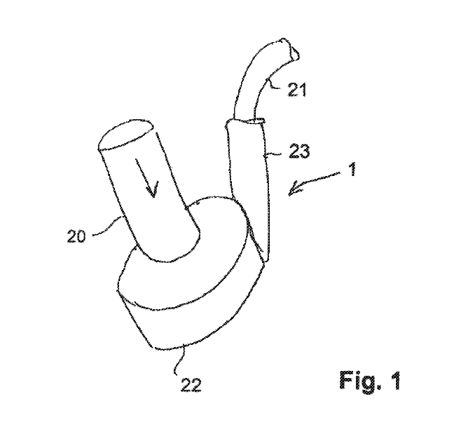

FIG. 1 shows an axial pump with tangential outlet in a perspective view from outside,

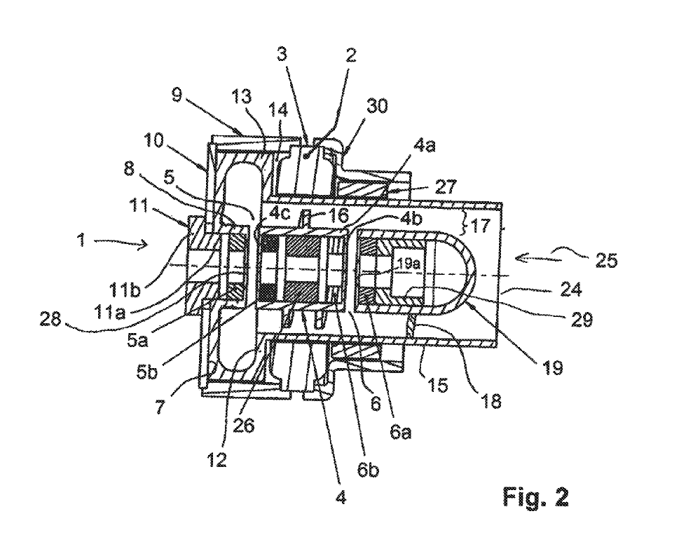

FIG. 2 shows a longitudinal section through an axial pump with tangential outlet similarly to that shown in FIG. 1, but without housing, and

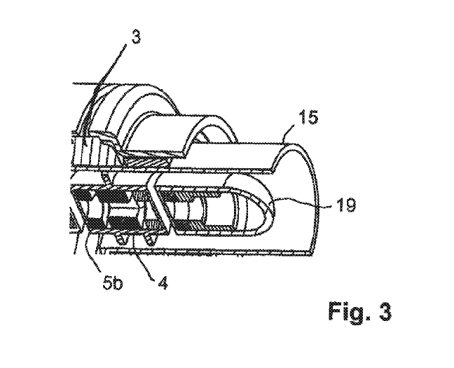

FIG. 3 shows a perspective view of a part (cut lengthwise) of the pump from FIG. 2, also without housing.

FIG. 1 shows a typical above-described pump, wherein the liquid is drawn in through the pump inlet 20. This is adjoined by the pump base 22, which has a flow chamber internally. The liquid is expelled again from the flow chamber via the tangentially outer pump outlet 23 in the direction of an outlet 21.

FIG. 1 for example is a pump as is used typically as a blood pump in patients intracorporeally (implanted in the body of the patient, a round hole being cut previously in the heart wall using a suitable tool during the implantation process), wherein blood for example is drawn in from a heart ventricle through a first cannula via the pump inlet 20 and is expelled again via a second cannula 21 in the direction of a blood vessel.

The pump 1 usually has an electric motor as drive, which is integrated in the pump and is controlled by means of a control unit (not shown in greater detail). The electric motor can be a brushless electric motor controlled by pulse width-modulated signals.

FIG. 2 shows a longitudinal section of the pump without housing, wherein the pump inlet opening 24 is arranged on the right-hand side, with liquid flowing in at said opening in the direction of the arrow 25. The liquid flows through the pump tube 15 through the annular conveying chamber 17 between the inner wall of the pump tube 15 and the hub of what is known as an inlet guide vane 19 (this is a flow guide vane, the hub of which is held radially in the middle in a spoke-like manner, wherein the spokes are formed as paddles and have flow-guiding properties) or between the inner wall of the pump tube 15 and the hub of the rotor 4, which rotor 4 adjoins the inlet guide vane 19 axially. The rotor 4 has conveying elements 16, for example in the form of blades extending helically, which convey the liquid in the axial direction 25. In addition, the liquid is accelerated in the axial direction and delivered to the flow chamber 13. Secondarily, the liquid is also accelerated tangentially, so that it is guided with the aid of the flow chamber 13 into the tangential pump outlet 23 shown in FIG. 1.

The drive motor, which has a radially outer stator 2 and a radially inner rotor 4, is integrated in the pump structure. The magnet gap of the electric motor lies here in the annular conveying chamber 17, through which the liquid also flows.

Reference sign 26 denotes the rotor magnets, which interact with the stator field in order to drive the pump.

The rotor has a cylindrical housing, which has a tube part 4a and two closure covers 4b, 4c. These seal the interior of the rotor with respect to the liquid to be conveyed. The inlet guide vane 19 also has a housing, which is closed by a cover 19a.

The pump arrangement has two axial bearings 5, 6, which are arranged at the two mutually opposed axial ends of the rotor 4. Each of the axial bearings 5, 6 has a stationary magnetically active part 5a, 6a, in particular a stationary magnet, and a magnetically active part 5b, 6b disposed in the rotor and possibly also formed as a magnet, but also possibly formed as a component guiding the magnetic flux in an effective manner. The stationary bearing magnets 5a, 6a both each attract the rotor magnet facing them, wherein the force is dependent on the distance. As a result, a stable axial position is not provided, and the magnetic bearing should be continually readjusted. Minimal shifts away from the desired position without readjustment would result in the rotor being moved further away from said position in the direction of one of the axial bearings.

A control system with a control coil 27 is provided, which lies in the magnetic flux circuit of the magnetic bearing flux of the axial bearing 6 or influences the magnetic flux and in the present case can make the magnetic flux of the axial bearing 6 stronger or weaker. The position of the rotor is set by the control system in such a way that the control coil 27 holds the rotor axially in a position intended therefor between the stationary bearing elements 5a, 6a. In order to reliably reach a favourable, compensated position of the rotor 4 during construction and at the time of assembly of the pump, it is important that the components of the pump that influence the magnetic fluxes can be assembled together in a predictable and reproducible way. In particular, the magnetic flux properties of the bearing magnets 5a, 5b of the laminated core 3, of the positioning element 9, and of the disc-like element 10 must be reproducible with sufficient accuracy on the side of the bearing 5.

In particular, the distance of the laminated core 3 from the stationary bearing magnet 5a in the axial direction 25 must also be established as accurately as possible. For this reason, the design of the pump is such that the laminated core 3 rests directly on the positioning element 9 in the axial direction without gaps, and the latter rests on the disc-like element 10, likewise without gaps, more specifically on the reference surface 7, which faces the pump interior and on which the flow chamber 13 also rests directly with the bearing magnet 5a distanced in a fixed manner from the reference surface.

The stationary bearing magnet 5a is integrated in the flow chamber 13, more precisely in an inwardly protruding connection piece 8 of the housing, i.e. it is fixed, for example moulded therein. A cover 28 is arranged above the bearing magnet 5a and separates the bearing magnet from the interior of the flow chamber 13. Here, the cover can also be fixedly integrated in the housing. The two bearing magnets 5a, 5b are thus separated from one another by the two covers 4c, 28 and the resultant axial bearing gap.

So that the laminated core 3 also comes to lie directly and without gaps on the positioning element 9, an assembly gap 14 is provided between the potting of the stator 2 and the housing of the flow chamber 13.

When the pump is commissioned after assembly, the control system of the control coil 27 can be put into operation first, with the rotor not turning.

In order to correct this starting position, an adjustment piece in the form of a connection piece 11 can be provided in a central opening of the disc-like element 10 in the direct vicinity of the stationary bearing magnet 5a. The connection piece 11 then has a defined distance (for example air gap) by its length or the length of its tube element 11a from the bearing magnet 5a, wherein the flange element 11b can rest directly on the disc-like element 10. The connection piece 11 consists of a material that guides the magnetic flux effectively, so that the magnetic flux can be influenced at this point by changing the distance between the connection piece 11 and bearing magnet 5a. However, an adjustment device can also be provided in the form of a thread between the connection piece 11 and the disc-like element 10, via which the connection piece 11 can be adjusted in the axial direction. The set position of the rotor 4 can be established for example via eddy current sensors or other distance sensors.

The optimised shaping of the reflux parts of the magnetic bearing also has an influence on the force necessary to initially break free the rotor, by an impulse of the control coil 27, from one of the axial bearing magnets to which the rotor had been attracted. By means of a suitable adjustment of the flux, the voltage pulse in the control coil necessary for this purpose can also be limited.

At the inlet-side end of the rotor 4, this comprises the rotating bearing magnet 6b, which interacts with the stationary bearing magnet 6a in the inlet guide vane 19. A flux-guiding element 29 is additionally provided in the inlet guide vane 19 axially behind the bearing magnet 6a, with the flux from the reflux element 30 disposed radially outside the pump tube 15 entering said flux-guiding element. This reflux element 30 guides the flux from the laminated core 3 axially in the direction of the inlet, from there radially inwardly via the control coil to the flux-guiding element 29, through the stationary bearing magnet 6a, the rotating bearing magnet 6b, the drive magnet 26, and back into the laminated core 3.

The inlet guide vane 19 is held in the pump tube by means of the spoke-like ribs 18, so that the liquid to be conveyed in the annular conveying chamber 17 is opposed by a defined resistance, which in the ideal scenario leads to favourable flow conditions.

1. A motor for a pump having comprising:
a radially outer stator and a radially inner rotor, the radially outer stator having a laminated core, wherein the rotor is mounted in two magnetic axial bearings distanced from one another in an axial direction of the rotor, wherein a position of the stator relative to a reference surface, against which a component rests, is established exclusively by a single positioning element arranged between the laminated core of the stator and the reference surface, wherein a stationary part of a first magnetic axial bearing of the two magnetic axial bearings is integrated in the component.

2. The motor according to claim 1, wherein the positioning element is configured to guide a magnetic reflux from the stator laminated core to the stationary part of a first magnetic axial bearing of the two magnetic axial bearings.

3. The motor according to claim 2, wherein the positioning element configured to guide the magnetic reflux of the first magnetic axial bearing rests against the laminated core of the stator and against the reference surface without gaps.

4. The motor according to claim 1, wherein the first magnetic axial bearing includes a first stationary magnetically active element configured to interact magnetically with a first magnetically active element of the rotor,
wherein a magnetic circuit is configured to be closed by the first magnetically active element of the rotor via the first stationary magnetically active element, a disc-like magnetically active element that forms the reference surface, and the positioning element, wherein the position element comprises a hollow-cylindrical magnetically active positioning element configured to guide magnetic flux between the stator and to the rotor, and
wherein the laminated core of the stator rests in the axial direction, without gaps, against the hollow-cylindrical magnetically active positioning element, and the hollow-cylindrical magnetically active positioning element rests in the axial direction, without gaps, against the reference surface of the disc-like magnetically active element.

5. The motor according to claim 1, wherein a central magnetically active connection piece extends in a direction of a first stationary magnetically active element and is positioned along a disc-like magnetically active element comprising the reference surface.

6. The motor according to claim 1, wherein a magnetically active connection piece is located between a disc-like magnetically active element, which forms the reference surface, and a first stationary magnetically active element, wherein the magnetically active connection piece comprises a tube element and a flange element configured to rest against the disc-like magnetically active element without play.

7. The motor according to claim 1, wherein one or more assembly gaps are defined between the stator and elements of a pump housing.

8. The motor according to claim 1, wherein a disc-like magnetically active element, which forms the reference surface, has a diameter that corresponds approximately to an outer diameter of the stator or that is between an inner diameter and an outer diameter of the stator.

9. The motor according to claim 1, wherein the position element comprises a hollow-cylindrical magnetically active positioning element, wherein the hollow-cylindrical magnetically active positioning element surrounds a flow chamber adjoining the stator axially.

10. The motor according to claim 1, wherein a stationary magnet of the first magnetic axial bearing is fixed relative to a pump housing.

11. A pump comprising a motor according claim 1, wherein the rotor carries conveying elements in a radial gap defined between the rotor and the stator for conveying a fluid.

12. A method, comprising:
placing reflux parts and a radially outer stator of a motor into a pump housing for a pump, the stator having a laminated core, the motor further including a radially inner rotor, wherein the rotor is mounted in two magnetic axial bearings distanced from one another in an axial direction of the rotor, wherein a position of the stator relative to a reference surface, against which a component rests, is established exclusively by a single positioning element arranged between the laminated core of the stator and the reference surface, wherein a stationary part of a first magnetic axial bearing of the two magnetic axial bearings is integrated in the component.
stabilizing the rotor by control of at least one magnetic axial bearing; and
calibrating the motor based on a stabilized position of the rotor.

13. The method according to claim 12, that wherein calibrating the motor further comprises:
introducing a magnetically active connection piece into a disk-like magnetically active element to modify a starting position of the rotor.

14. A group of pumps, each comprising a motor, the motor comprising a radially outer stator and a radially inner rotor, the radially outer stator having a laminated core, wherein the rotor is mounted in two magnetic axial bearings distanced from one another in an axial direction of the rotor, wherein a position of the stator relative to a reference surface, against which a component rests, is established exclusively by a single positioning element arranged between the laminated core of the stator and the reference surface, wherein a stationary part of a first magnetic axial bearing of the two magnetic axial bearings is integrated in the component wherein at least two of the pumps have different magnetically active connection pieces.

15. The method of claim 12 wherein the pump comprises an intracorporeal heart pump.

16. The method of claim 12, wherein calibrating the pump based on the stabilized position of the rotor further comprises calibrating, when the motor is not turning, the motor based on an axial direction corresponding to the magnetic axial bearing.

17. The method of claim 12, wherein calibrating the pump based on the stabilized position of the rotor further comprises replacing a connection piece with a magnetically active connection piece to modify a starting position of the rotor.

18. The motor according to claim 7, wherein the elements of the pump housing comprise delimitation elements of a flow chamber adjoining the stator axially.

19. The motor according to claim 10, wherein the stationary magnet of the first magnetic axial bearing is secured to the pump housing.
